(19) United States
(12) Patent Application Publication
(10) Pub. No.: US 2021/0403858 A1
(43) Pub. Date: Dec. 30, 2021

(54) Bacillus strain and microbial preparation of bacillus and method of using microbial preparation

(71) Applicant: ROLLING GREENS CO., LTD., TAICHUNG CITY (TW)

(72) Inventor: Po-Yu Chen, Taichung City (TW)

(21) Appl. No.: 16/916,151

(22) Filed: Jun. 30, 2020

(51) Int. Cl. C12N 1/20; A01N 63/20

(52) U.S. Cl. CPC C12N 1/20; C12R 1/07; A01N 63/20

(57) ABSTRACT

A Bacillus strain is a RG BV-1 strain in Bacillus velezensis, and a base sequence of the RG BV-1 strain has a serial number 1 which is presented by 16S rDNA. The RG BV-1 strain of the Bacillus velezensis is capable of resolving chelated phosphorus in soils to phosphorus absorbed by plants, thus promoting a plant growth and avoiding diseases and pests. A microbial preparation consisting of the Bacillus strain and/or cultures of the Bacillus strain is active ingredients of the microbial preparation.

### FIELD OF THE INVENTION

The present invention relates to a Bacillus strain and a microbial preparation of Bacillus and a method of using the microbial preparation which are capable of resolving chelated phosphorus in soils to phosphorus absorbed by plants, avoiding diseases and pests, promoting a plant growth and microbial proliferation in the soils, and reducing application dose of chemical phosphate fertilizer.

### BACKGROUND OF THE INVENTION

Since Staltrom discovered phosphorus-dissolving microorganisms in the soil in 1903, researchers have begun various studies, such as types, phosphorus dissolution mechanism, and application effect of phosphate-dissolving bacteria.

According to incomplete statistics, a total of 36 genera and 89 species of phosphorus-dissolving microorganisms have been screened worldwide, including 27 species of fungi, 58 species of bacteria, and 4 species of actinomycetes. Bacillus sp. Sheathella and Penicillium. It is generally believed that the phosphorus-dissolving mechanism of phosphorus-soluble strains is related to the production of low-molecular-weight organic acids. Organic acids chelate metal ions on insoluble phosphates through hydroxyl groups and hydroxyl groups, thereby converting insoluble phosphorus into soluble phosphorus.

However, the dissolution of poorly soluble phosphorus by phosphate-dissolving bacteria is a complex phenomenon, such as nutrients, physiology, and growing culture conditions all affect it.

The present invention has arisen to mitigate and/or obviate the afore-described disadvantages.

### SUMMARY OF THE INVENTION

The primary objective of the present invention is to provide a Bacillus strain and a microbial preparation of Bacillus and a method of using the microbial preparation which are capable of resolving chelated phosphorus in soils to phosphorus absorbed by plants, avoiding diseases and pests, promoting a plant growth and microbial proliferation in the soils, and reducing application dose of chemical phosphate fertilizer.

To obtain above-mentioned objective, a Bacillus strain provided by the present invention is a RG BV-1 strain in Bacillus velezensis, and a base sequence of the RG BV-1 strain has a serial number 1 which is presented by 16S rDNA. The RG BV-1 strain of the Bacillus velezensis is capable of resolving chelated phosphorus in soils to phosphorus absorbed by plants, thus promoting a plant growth and avoiding diseases and pests.

A microbial preparation consisting of the Bacillus strain and/or cultures of the Bacillus strain is active ingredients of the microbial preparation.

### BRIEF DESCRIPTION OF THE DRAWINGS

FIG. 1 is a chart showing the application of a Bacillus strain and a microbial preparation of Bacillus according to a preferred embodiment of the present invention.

FIG. 2 is a chart showing another application of the Bacillus strain and the microbial preparation of the Bacillus according to the preferred embodiment of the present invention.

FIG. 3 is another chart showing another application of the Bacillus strain and the microbial preparation of the Bacillus according to the preferred embodiment of the present invention.

FIG. 4 is a chart showing the experiment of the Bacillus strain and the microbial preparation of the Bacillus according to the preferred embodiment of the present invention.

### DETAILED DESCRIPTION OF THE PREFERRED EMBODIMENTS

A Bacillus strain and a microbial preparation of Bacillus and a method of using the microbial preparation according to a preferred embodiment of the present invention, the Bacillus strain is a RG BV-1 strain in Bacillus velezensis, and a base sequence of the RG BV-1 strain has a serial number 1 which is presented by 16S rDNA.

Accordingly, the RG BV-1 strain of the Bacillus velezensis is capable of resolving chelated phosphorus in soils to phosphorus absorbed by plants, thus promoting a plant growth and avoiding diseases and pests.

The RG BV-1 strain or cultures of the RG BV-1 strain are active ingredients of the microbial preparation.

The microbial preparation is in a form of any one of powders, solution, suspensions, and granules, wherein the microbial preparation is any one of plant disease control agent, nematode control agent, and plant growth promoter.

The microbial preparation further consists of fertilizer additives having organic fertilizer or microbial fertilizer, and the organic fertilizer or the microbial fertilizer consists of any one of amino acids, seaweed extract, humic acid, bird droppings, molasses, bone meal, fish essence, seaweed powders, sawdust, lime, oil meal, rice bran, and grass ashes. Alternatively, the organic fertilizer or the microbial fertilizer consists of a mixture of at least two of amino acids, seaweed extract, humic acid, bird droppings, molasses, bone meal, fish essence, seaweed powders, sawdust, lime, oil meal, rice bran, and grass ashes.

Taking rice and lettuce for example, IAA sensitivity of the microbial preparation is tested in steps of:

disinfecting and germinating rice seedlings;

putting the rice seedlings in a sterilized Petri dish in which a filter paper is disposed;

adding 3 mL of 0.03 μM, 3 μM, 30 μM, and 300 μM of IAA aqueous solutions respectively;

moving the sterilized Petri dish into a plant growth box for 4 days;

measuring an average height of buds of the rice seedlings and an average height of roots of the rice seedlings;

disinfecting seeds of the lettuce with 5% sodium hypochlorite and putting the seeds of the lettuce in another sterilized Petri dish in which a filter paper is disposed;

adding 3 mL of 0.03 μM, 3 μM, 30 μM, and 300 μM of IAA aqueous solutions respectively;

moving another sterilized Petri dish into another plant growth box for 3 days; and

measuring a germination rate and a height of each lettuce and a length of a root of each lettuce.

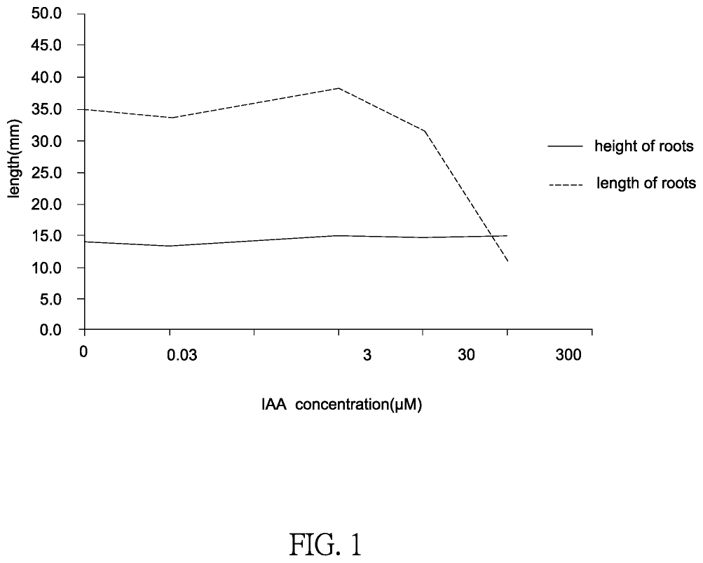

Accordingly, as shown in FIG. 1, a low concentration of IAA (i.e. 0.03 μM to 30 μM) does not promote a growth of the roots of the rice seedlings.

However, when a concentration of IAA is 300 μM, the growth of the roots of the rice seedlings is limited. Thus, the roots of the rice seedlings do not grow obviously when the concentration of IAA is within 0.00003 μM to 0.003 μM.

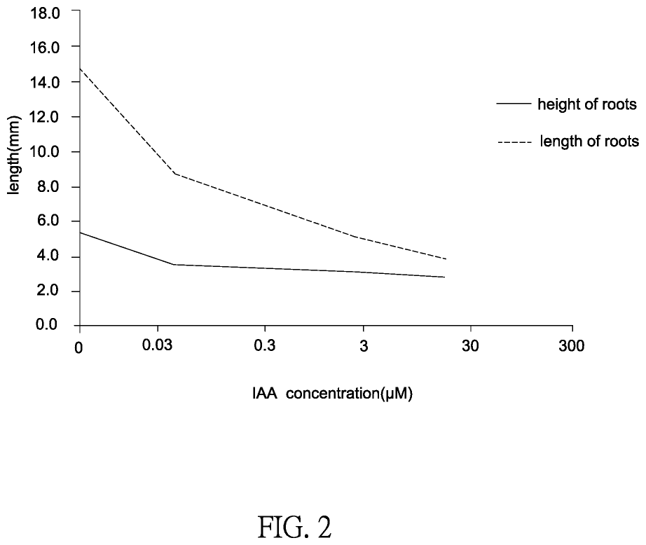

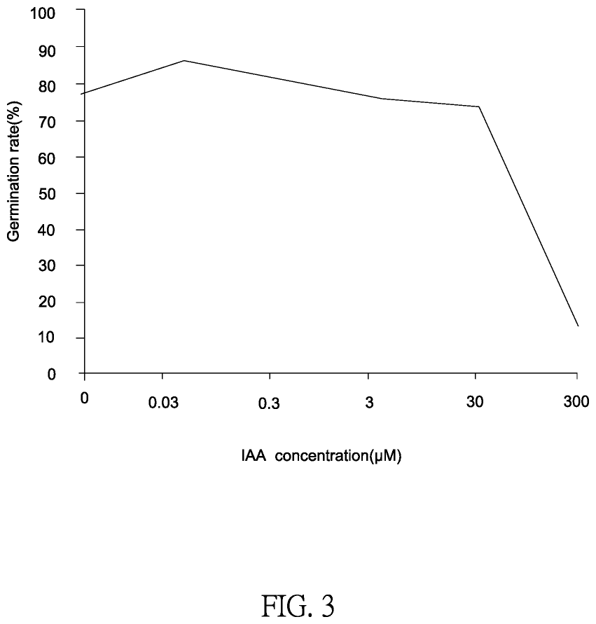

Referring to FIGS. 2 and 3, when IAA is not provided in this embodiment, a height of lettuce sprouts is 5.3 mm, and a length of lettuce roots is 14.8 mm. When the lowest concentration of IAA is 0.03 μM, the height of the lettuce sprouts is 3.5 mm, and the length of the lettuce roots is 8.8 mm, hence the lowest concentration of IAA reduces a growth of the lettuce seedlings. When the concentration of IAA is 300 μM, the germination rate of seeds of the lettuce is influenced. When no IAA is provided, the germination rate of the seeds of the lettuce is 78%. When the concentration of IAA is 300 μM, the germination rate of the seeds of the lettuce is 13%. Accordingly, IAA secreted from the Bacillus strain restrains the growth of dicotyledonous, thus obtaining herbicide to the dicotyledonous.

In use, when the microbial preparation is the solution or suspended particles, it is sprayed or inoculated to seeds or roots of plant seedlings in the soils. Alternatively, the microbial preparation is poured to the soils so that the seeds or the roots of the plant seedlings contact with the microbial preparation.

A method of producing the microbial preparation comprises steps of:

a) cultivating the Bacillus strains, wherein 10 g soils are put into 100 ml sterile water so as to produce soil diluent in plate dilution manner, the soil diluent is coated on Pikovskaya (PVK) medium, and the PVK medium is cultivated for 48 hours in a temperature of 30° C., colonies having dissolved phosphorus are picked from the Bacillus strains, then the Bacillus strains are purified and preserved within 4° C. and −80° C. Phospholytic bacteria of corn roots are separated from the soils by ways of the PVK medium and are subcultured repeatedly so as to acquire a selected Bacillus strain having stable dissolved phosphorus;

b) cultivating IAA secretion of the selected Bacillus strain, wherein the selected Bacillus strain is inoculated to 100 mL Lysogeny broth (LB) medium consisting of 100 mg/L Tryptophan concentration, and the selected Bacillus strain is shocked and cultivated for 24 hours in a temperature of 30° C., 5 mL bacterial solution is acquired and is centrifuged in 9000 g for 15 minutes, and 1 mL supernatant and 1 mL reaction reagent (such as Salkowsky reagent) are mixed and has a negative phototropism for 1 hour, thereafter absorbance at 540 nm is measured, and a compared supernatant having 8.2 mg/L concentration is obtained by using a standard curve line;

c) obtaining nitrogen fixation of the selected Bacillus strain, wherein the selected Bacillus strain is coated on a nitrogen-free medium, when the selected Bacillus strain has the nitrogen fixation, nitrogen is reduced to ammonium nitrogen so that a color of the nitrogen-free medium is transformed from green into blue, in other words, the nitrogen-free medium is cultivated for two weeks to have blue color, hence the selected Bacillus strain has the nitrogen fixation after being tested and growing in the nitrogen-free medium, wherein the color of the nitrogen-free medium is transformed from the green into the blue; and

d) obtaining dissolved phosphorus of a purified Bacillus strain, wherein the purified Bacillus strain is coated in a Pikovskaya agar medium, when the purified Bacillus strain has dissolved phosphorus, solid calcium phosphate is dissolved in the Pikovskaya agar medium by the dissolved phosphorus of the purified Bacillus strain so that the Pikovskaya agar medium is translucent, when a transparent ring of the dissolved phosphorus presents around the purified Bacillus strain after cultivating the purified Bacillus strain for 7 days, the dissolved phosphorus of the purified Bacillus strain is negative after being tested, and a transparent phosphor ring does not form in the calcium phosphate medium.

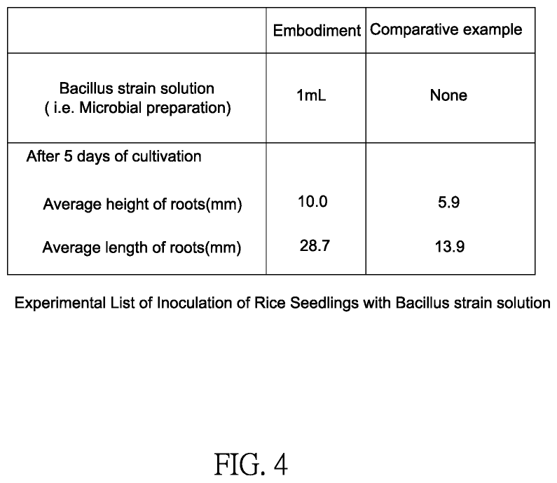

The Bacillus strain is inoculated to the rice seedlings. Taking Tainan No. 12 of rice seedlings for example, the rice seedlings are heated up to 62° C. for 15 minutes to sterilize pathogenic fungi of the rice seedlings, and the rice seedlings are socked in a water of the growth box water in a temperature of 40° C. so as to avoid lights and to sprout, wherein the filter paper is put into the sterilized Petri Dish, and 15 rice seedlings are put into the sterilized Petri Dish, wherein a length of roots of the 15 rice seedlings is less than 1.0 cm, thereafter 1 mL bacteria strain solution is centrifuged to remove culture solution, wherein 0.85% sodium chloride solution are applied to suspend the bacteria, and the 0.85% sodium chloride solution are added into the sterilized Petri Dish so as to test the 15 rice seedlings by ways of the filter paper, wherein the concentration of the bacterial solution is more than 106 CFUm/L. The sterilized Petri Dish is moved into the growth box, and an average height of the buds of the 15 rice seedlings and an average length of the roots of the 15 rice seedlings are measured after 4 days. A condition of the growth box is light photoperiod L: dark photoperiod D is 12 hours: 12 hours, a temperature is 28° C., and a relative humidity is 72° C. Referring to FIG. 4, the average height of the buds of the 15 rice seedlings is 10.2 mm, and the average length of the roots of the 15 rice seedlings is 28.7 mm.

In a comparative example, the Bacillus strain is not inoculated to the rice seedlings, the rice seedlings are heated to 62° C. for 15 minutes to sterilize pathogenic fungi of the rice seedlings, and the rice seedlings are socked in the water of the growth box water in the temperature of 40° C. so as to avoid lights and to sprout, wherein the filter paper is put into the sterilized Petri Dish, and 15 rice seedlings are put into the sterilized Petri Dish, wherein a length of roots of the 15 rice seedlings is less than 1.0 cm, thereafter 0.85% saline solution is applied to cultivate the 15 rice seedlings, and the sterilized Petri Dish is moved into the growth box, wherein an average height of the buds of the 15 rice seedlings and an average length of the roots of the 15 rice seedlings are measured after 4 days. A condition of the growth box is light photoperiod L: dark photoperiod D is 12 hours: 12 hours, a temperature is 28° C., and a relative humidity is 72° C. Referring to FIG. 4, the average height of the buds of the 15 rice seedlings is 5.9 mm, and the average length of the roots of the 15 rice seedlings is 13.9 mm.

The average height of the buds and the average length of the roots of the 15 rice seedlings of the embodiment and the comparative example are analyzed statistically at 95% confidence level of t-test. Accordingly, the length of the roots of the 15 rice seedlings of the embodiment is more than that of the comparative example, thus promoting growth of the rice seedlings by using the Bacillus strain of the present invention.

Accordingly, the Bacillus strain and the microbial preparation are capable of resolving the chelated phosphorus in the soils to the phosphorus absorbed by the plants, thus promoting the plant growth and avoiding the diseases and pests.

While the preferred embodiments of the invention have been set forth for the purpose of disclosure, modifications of the disclosed embodiments of the invention and other embodiments thereof may occur to those skilled in the art. Accordingly, the appended claims are intended to cover all embodiments which do not depart from the spirit and scope of the invention.

What is claimed is:

1. A Bacillus strain being a RG BV-1 strain in Bacillus velezensis, and a base sequence of the RG BV-1 strain having a serial number 1 which is presented by 16S rDNA.

2. The Bacillus strain as claimed in claim 1, wherein the RG BV-1 strain of the Bacillus velezensis is capable of resolving chelated phosphorus in soils to phosphorus absorbed by plants, thus promoting a plant growth and avoiding diseases and pests.

3. A microbial preparation consisting of the Bacillus strain and/or cultures of the Bacillus strain of claim 1 which is active ingredients of the microbial preparation.

4. The microbial preparation as claimed in claim 3, wherein the microbial preparation is any one of plant disease control agent, nematode control agent, and plant growth promoter.

5. The microbial preparation as claimed in claim 3, wherein the microbial preparation is in a form of any one of powders, solution, suspensions, and granules.

6. The microbial preparation as claimed in claim 3, wherein the microbial preparation contacts with seeds or roots of the plant seedlings.

7. The microbial preparation as claimed in claim 6, wherein the plant seedlings are rice.

8. The microbial preparation as claimed in claim 6, wherein the microbial preparation is inoculated to the seeds or the roots of plant seedlings in the soils.

9. The microbial preparation as claimed in claim 7, wherein the microbial preparation is inoculated to the seeds or the roots of plant seedlings in the soils.
